(19) United States
(12) Patent Application Publication
(10) Pub. No.: US 2012/0323133 A1
(43) Pub. Date: Dec. 20, 2012

(54) IDENTIFICATION OF CULPRIT CORONARY ARTERY USING ANATOMICALLY ORIENTED ECG DATA FROM EXTENDED LEAD SET

(75) Inventors: James M. Lindauer, San Francisco, CA (US); Sophia Huai Zhou, Briarcliff Manor, NY (US)

(73) Assignee: KONINKLIJKE PHILIPS ELECTRONICS N.V., EINDHOVEN (NL)

(21) Appl. No.: 13/522,889

(22) PCT Filed: Dec. 16, 2010
(86) PCT No.: PCT/IB10/55891

§ 371 Date: Sep. 6, 2012

Related U.S. Application Data
(60) Provisional application No. 61/296,886, filed on Jan. 20, 2010.

(51) Int. Cl. A61B 5/0452

(52) U.S. Cl. 600/523

(57) ABSTRACT

An ECG monitoring system analyzes ECG signals of leads associated with different anatomical locations of the body for evidence of ST elevation in the lead signals. The ST elevation and depression measurements of the leads are plotted in a graphical display organized in relation to the anatomical points which are the sources of the lead signals. In a polar graphical display format, each lead signal is plotted on its own anatomically-oriented axis to prevent conflict between multiple lead signals. In a linear or rectilinear graphical display format, each lead signal is plotted on its own row or column of the display. Missing lead signal values are filled in with averaged or interpolated values from other leads.

This application is a continuation-in-part of pending U.S. patent application no. [IB2008/055149, filed Dec. 8, 2008], filed Jan. 20, 2010, which claims the benefit of U.S. provisional application No. 61/014,613, filed Dec. 18, 2007.

This invention relates to electrocardiographic (ECG) monitoring systems and, in particular, to real-time ST monitoring system which automatically identify, by means of an anatomically-oriented presentation, a culprit coronary artery which has caused an acute myocardial infarction.

Electrocardiography (ECG) is in widespread use to produce records derived from voltages produced by the heart on the surface of the human body. The records so produced are graphical in character and require expert interpretation and analysis to relate the resulting information to the heart condition of the patient. Historically, such records have been produced directly as visible graphic recordings from wired connections extending from the subject to the recording device. With advances in computer technology, it has become possible to produce such records in the form of digitally stored information for later replication and analysis.

An emergency clinical application where ECG records are critical is the diagnosis of symptoms of acute coronary disease, commonly referred to as heart attacks. Patients with acute coronary syndrome (ACS) such as chest pain or discomfort and shortness of breath are often diagnosed electrocardiographically, where the elevation or depression of the ST segments of ECG waveforms are critically analyzed. One scenario that frequently occurs is that the ST elevation of a patient's ECG at the time of admission to an emergency department or a chest pain center of a hospital does not meet the diagnostic criteria for a definitive ST elevation myocardial infarct (STEMI) diagnosis. In such cases, patients are often connected to an ECG monitor for ST segment monitoring to observe the progression or regression of ST variation, particularly with patients with a history of acute coronary syndrome (ACS). If the patient's condition deteriorates, the clinical caregiver responsible for the patient needs to know the coronary artery and the region of the myocardium at risk before intervention can proceed.

Another scenario is that an ACS patient is definitively diagnosed with an ECG presentation of STEMI, and undergoes interventional reperfusion therapy. Proven therapies to restore myocardial reperfusion include thrombolytics or percutaneous coronary intervention to open the infarct-related artery. Coronary artery bypass graft (CABG) is another perfusion therapy often applied to ACS patients with more serious occlusions. After the interventional procedure and during the thrombolytic therapy, the patient is usually connected to an ECG monitor for ST monitoring and observation in a recovery room, intensive care unit (ICU) or cardiac care unit (CCU) for observation of regression or progression of the patient condition. New episodes of coronary artery occlusion may occur if the previously cleared coronary artery becomes clotted again or an occlusion occurs in a different artery or the ST deviation will return to normal when the patient's coronary perfusion is restored. Since the first sixty minutes are critical for salvage of the myocardium, it is critical that clinical personnel capture the recurrence episodes early to prevent further damage to the myocardium.

The ST monitoring commonly performed in these scenarios has limitations, however. Episodes with ST elevation or ST depression are often missed due to use of a limited number of electrodes. Hospitals have widely varying protocols for lead availability and lead systems used in ST monitoring. Some hospitals use one channel (3 wire) ECG monitors, some use three channel (5 wire) systems, while others use five channel (six wire) systems, or twelve leads derived from five or six channel systems or calculated from a direct recording of eight channels. ST monitor design is often not intuitive for general clinical caregivers who may not have adequate training to understand the relationship between ECG leads and the associated myocardial regions or coronary arteries. Numeric changes or waveforms of ST segments displayed on bedside monitors do not have indications of corresponding relationships between each lead and the myocardial region at risk. Accordingly improved ECG monitors and protocols would improve the standard of care in these situations.

An ECG monitoring system which provides improved care in these situations is described in U.S. provisional patent application Ser. No. 60/954,367 entitled “AUTOMATED IDENTIFICATION OF CULPRIT CORONARY ARTERY (Zhou et al.), filed Aug. 7, 2007. The ECG monitor described in this patent application analyzes the ST segments of ECG waveforms produced by leads associated with different regions of the body. On the basis of the ST elevation and depression exhibited by different groups of leads, the system identifies to a clinician the coronary artery which is the likely location of an occlusion, the “culprit” coronary artery. The system does this using standard ECG lead placement and multiple ECG waveform presentation. While such a display provides all of the relevant diagnostic information for a definitive diagnosis, including an indication of the culprit artery, significant skill in the interpretation of ECG waveforms is still necessary to relate the ECG data to the culprit artery indicated by the system. It would be desirable to have an unambiguous, graphical way of relating the ECG data to the diagnostic indication, so that the clinician could immediately appreciate the validity of the diagnostic determination before undertaking his or her own more detailed waveform analysis. The shorter the time to a definitive diagnosis, the sooner that myocardial perfusion can be restored, with less damage to the heart and a lower risk for heart failure or death.

In accordance with the principles of the present invention, an ECG monitoring system is described which acquires ECG waveforms from a plurality of leads and analyzes the ST segment elevation and depression present. This ST segment information is presented in a graphical display which displays the information in relation to the anatomy of the patient. In an illustrated embodiment, the graphical display presents ST segment information in both a vertical (frontal) and a horizontal (lateral) orientation in relation to the lead positions which produced the information. The anatomically-oriented display shows at a glance an indication of the culprit coronary artery and the size of the myocardial region with the infarct or injury. When used with extended leads, the extended lead information is graphically located so as to prevent conflict or ambiguity with the other leads in the graphical display. The anatomically-oriented display may be produced in real time during monitoring, with comparison to a baseline condition, or in a time-lapsed display which indicates progression of the condition.

In the drawings:

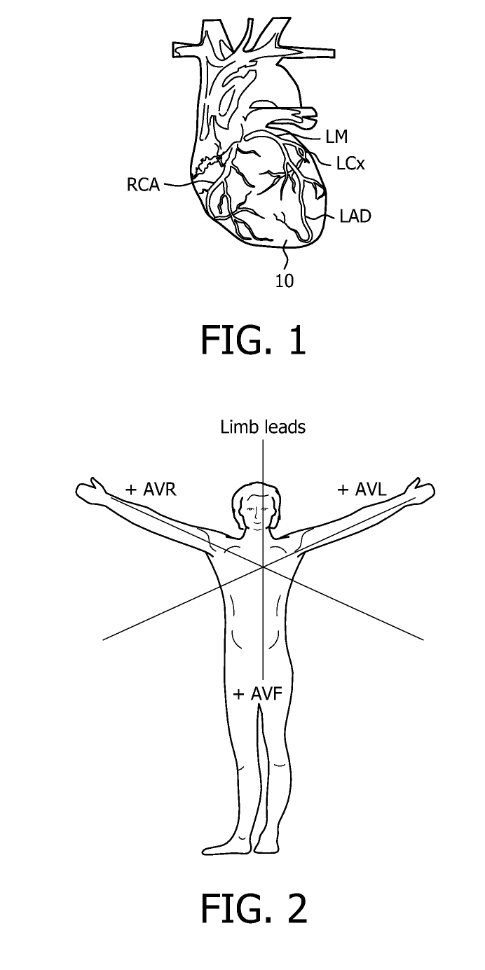

FIG. 1 is an anatomical illustration of the heart, showing the coronary arteries wrapping around the heart.

FIG. 2 is an illustration of the location of ECG limb leads in relation to a standing (vertical) individual.

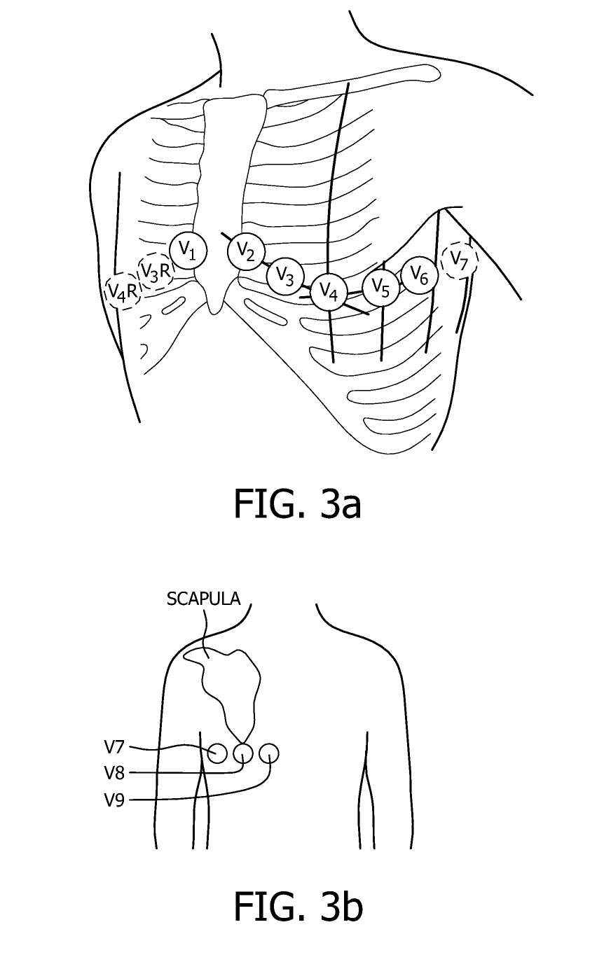

FIGS. 3a and 3b show standard chest electrode placement for an ECG exam.

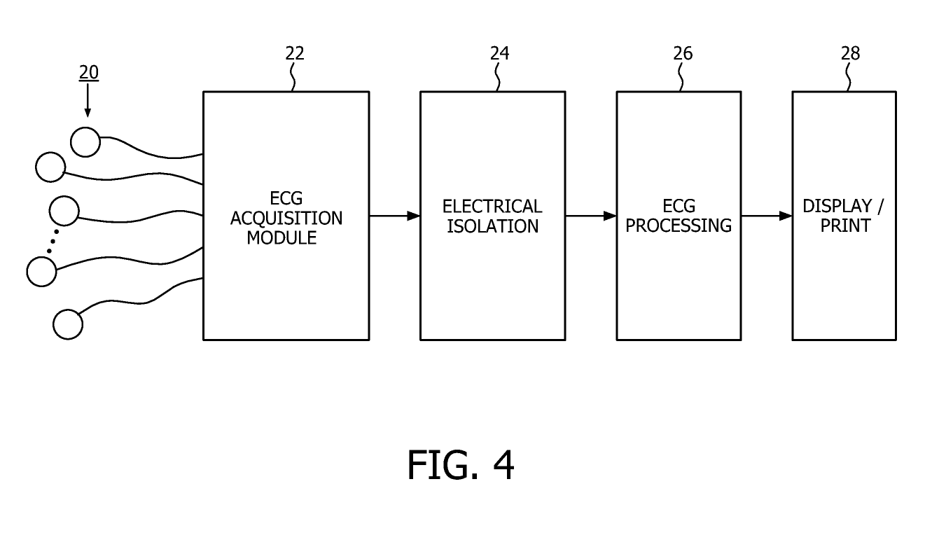

FIG. 4 is a block diagram of major subsystems of an ECG monitoring system suitable for use with the present invention.

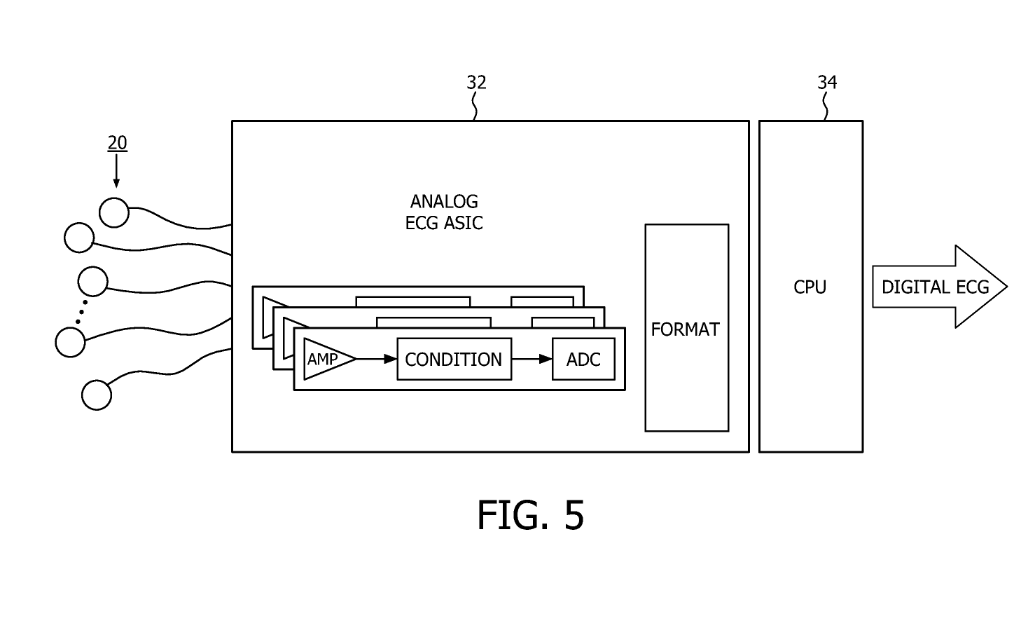

FIG. 5 is a block diagram of the front end of an ECG system.

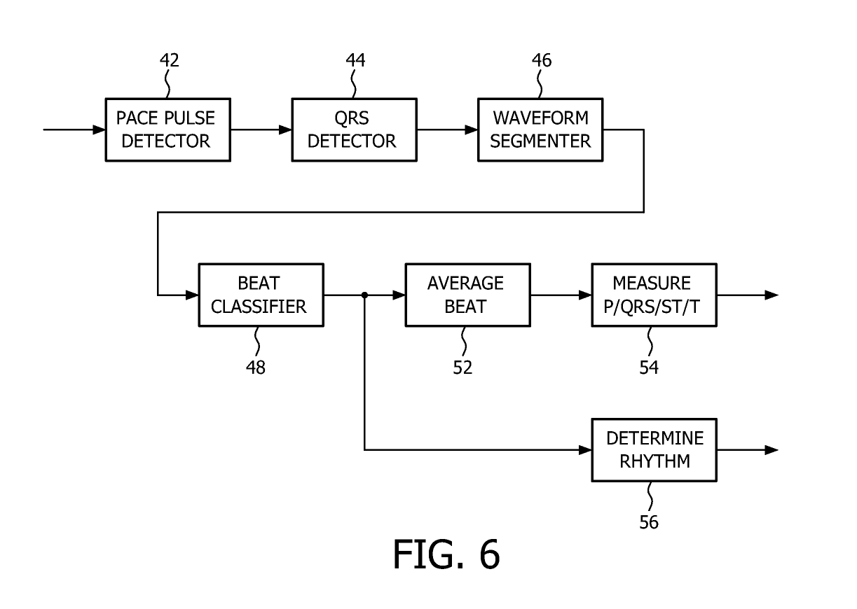

FIG. 6 is a block diagram of the processing module of a typical ECG monitor.

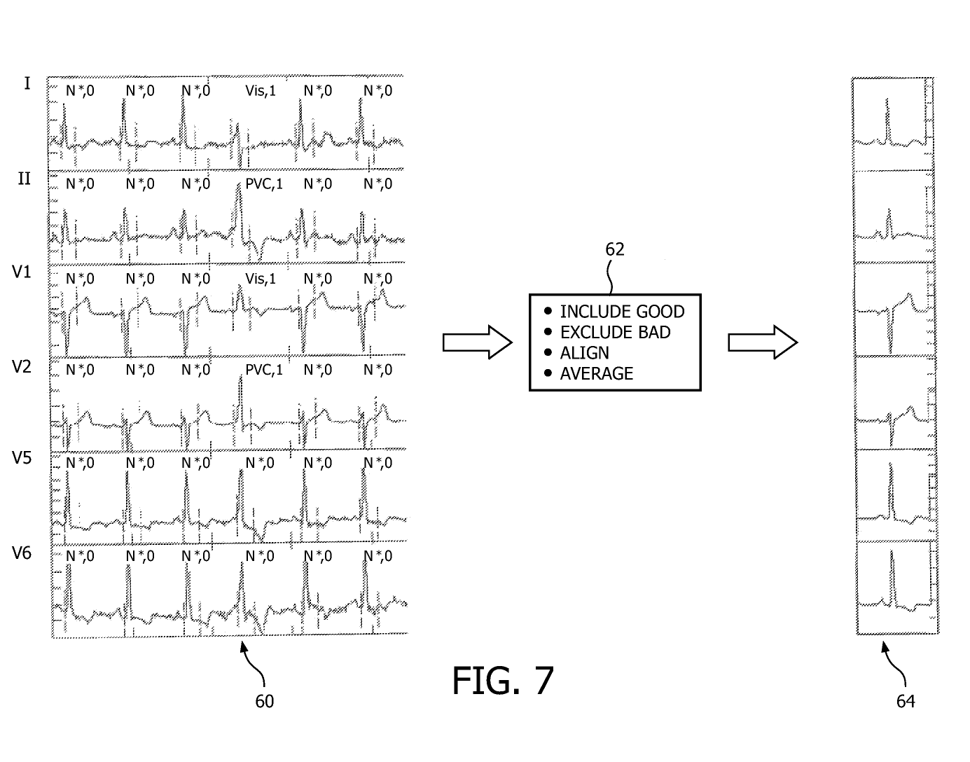

FIG. 7 illustrates the processing of ECG trace data to provide information about the heartbeat and its rhythm.

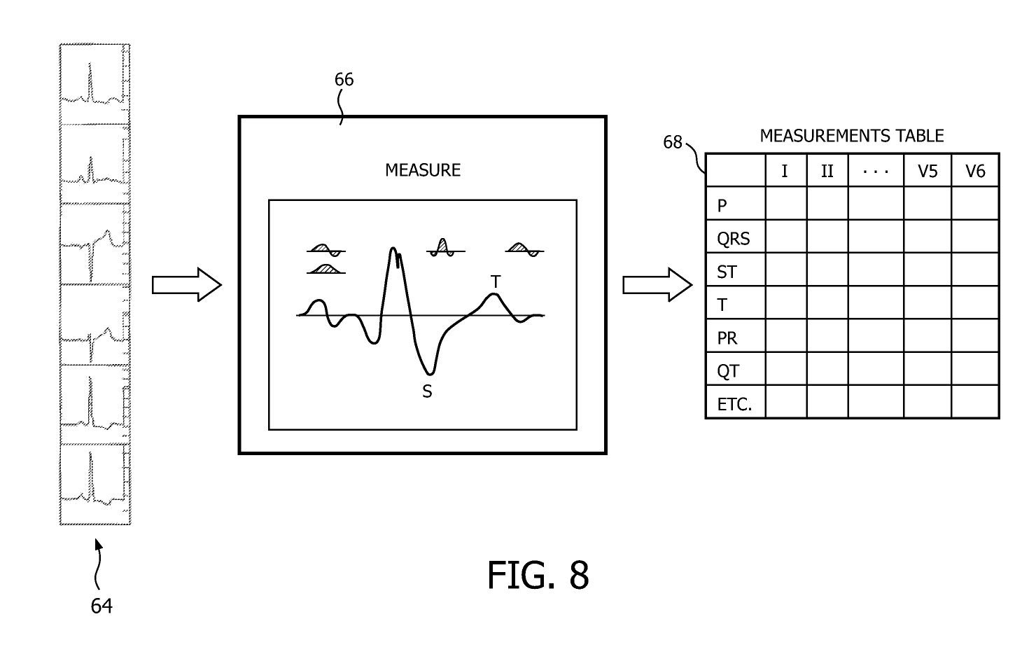

FIG. 8 illustrates the measurement of different parameters of an ECG trace.

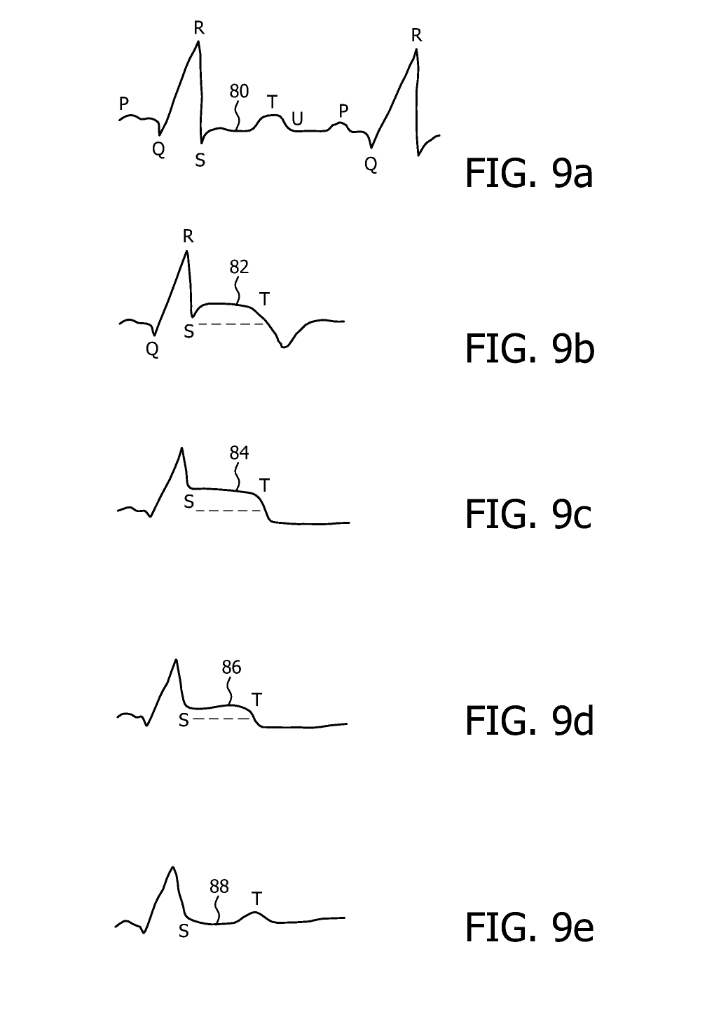

FIG. 9a illustrates the segments of a normal ECG signal.

FIGS. 9b-9e illustrate ECG traces with elevated and depressed ST segments which may be used to produce an anatomically-oriented graphical display for culprit coronary artery identification in accordance with the principles of the present invention.

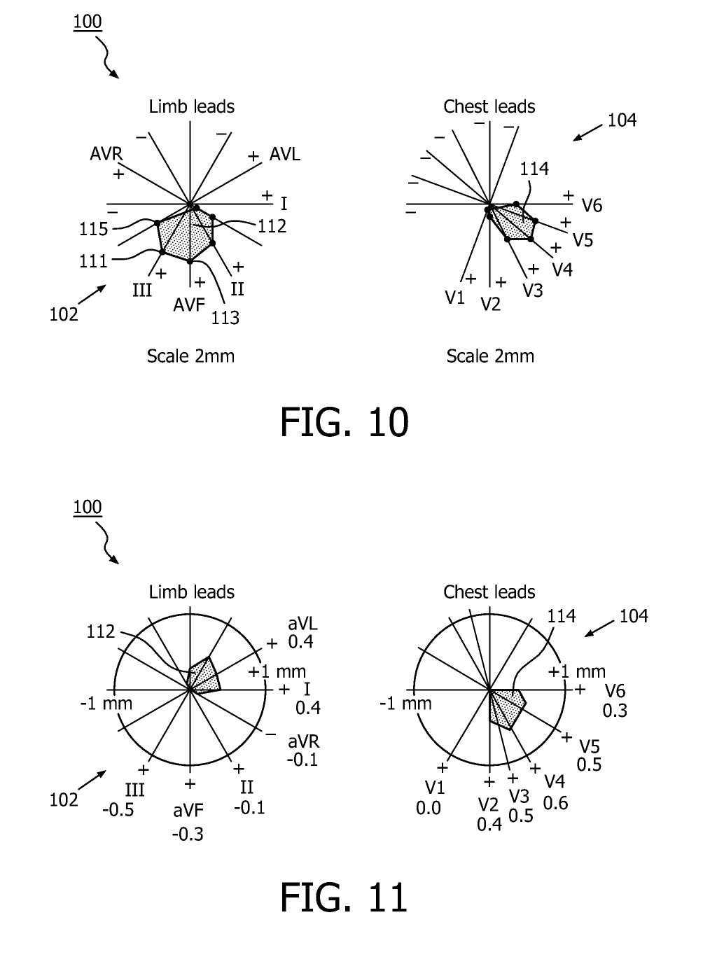

FIG. 10 illustrates an anatomically-oriented graphical display for culprit coronary artery identification in accordance with the principles of the present invention.

FIG. 11 illustrates a second anatomically-oriented graphical display, showing the ST segment values used to produce the display.

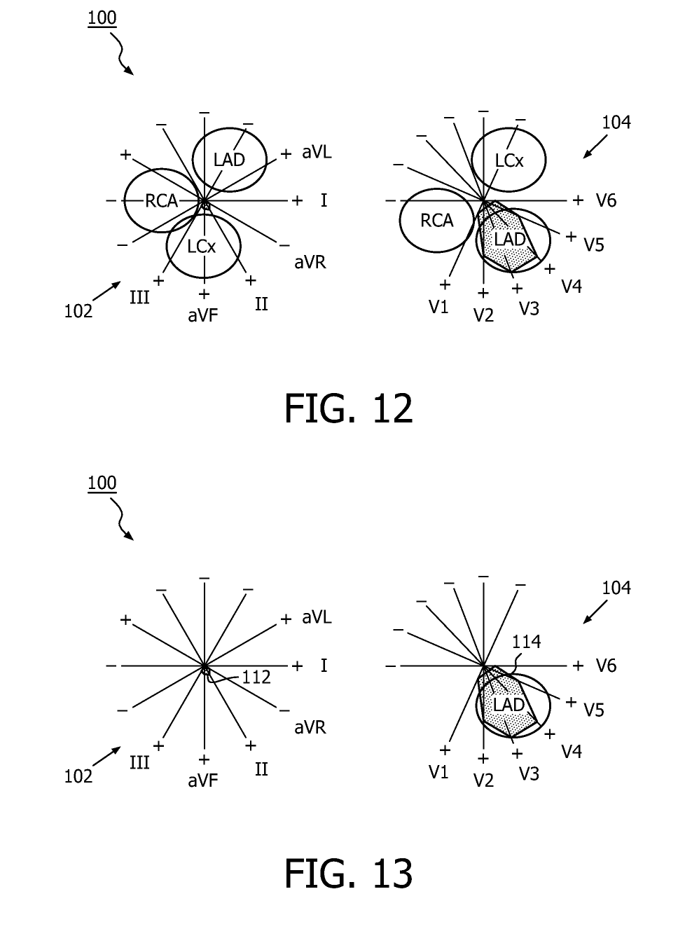

FIG. 12 illustrates the identification of a culprit coronary artery by means of an anatomically-oriented graphical display in accordance with the principles of the present invention.

FIG. 13 is an example of an anatomically-oriented graphical display of the present invention indicating occlusion of the left anterior descending (LAD) coronary artery.

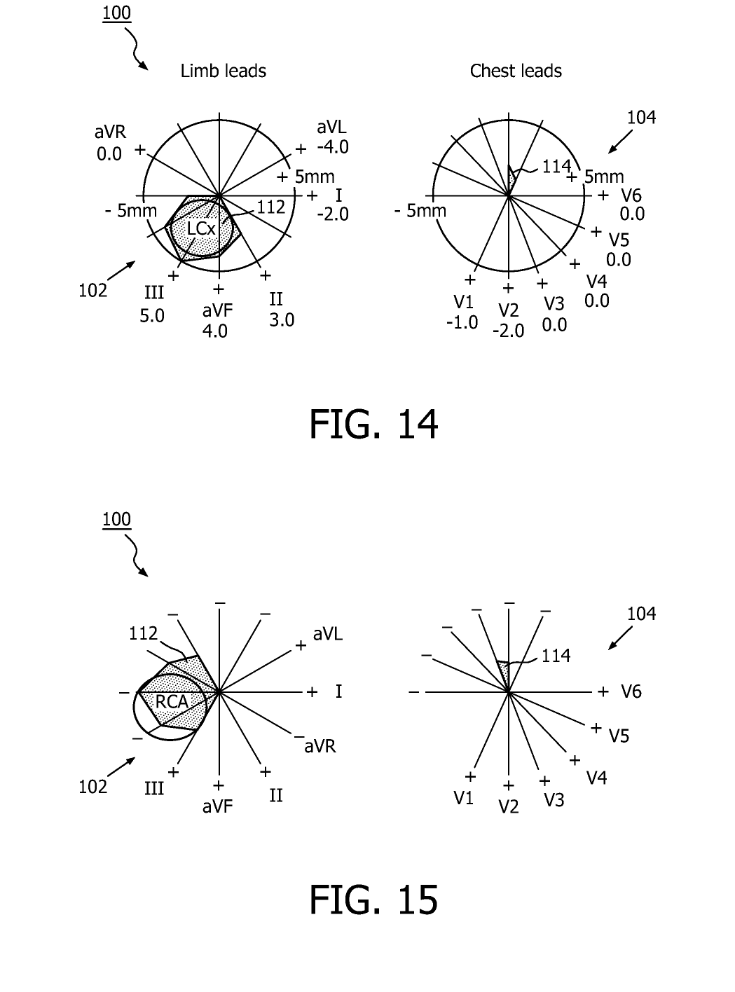

FIG. 14 is an example of an anatomically-oriented graphical display of the present invention indicating occlusion of the left circumflex (LCx) coronary artery.

FIG. 15 is an example of an anatomically-oriented graphical display of the present invention indicating occlusion of the right coronary artery (RCA).

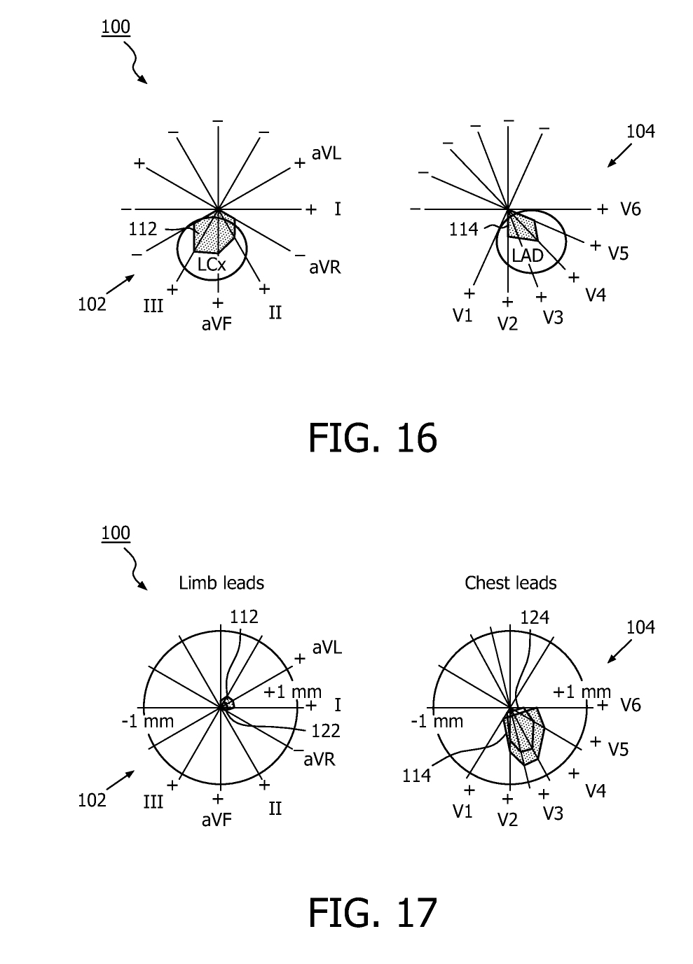

FIG. 16 is an example of an anatomically-oriented graphical display of the present invention indicating occlusion of both the left circumflex and the left anterior descending coronary arteries.

FIG. 17 illustrates an anatomically-oriented graphical display of the present invention in which the current ST elevation and depression characteristics are compared to baseline characteristics.

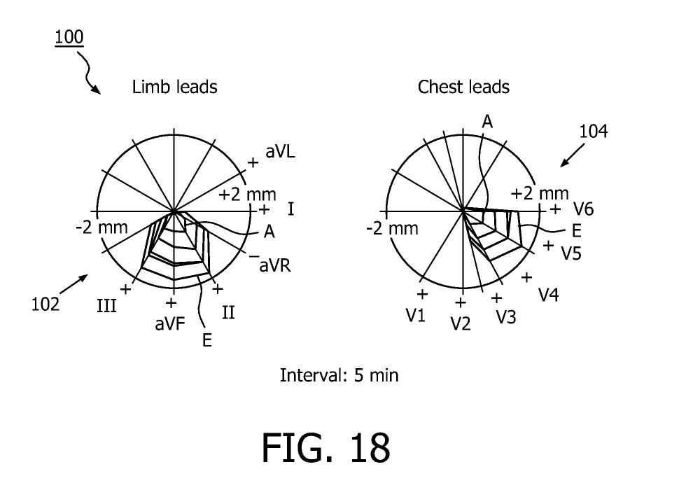

FIG. 18 illustrates an anatomically-oriented graphical display of the present invention in which the trend of ST elevation and depression characteristics over time is presented.

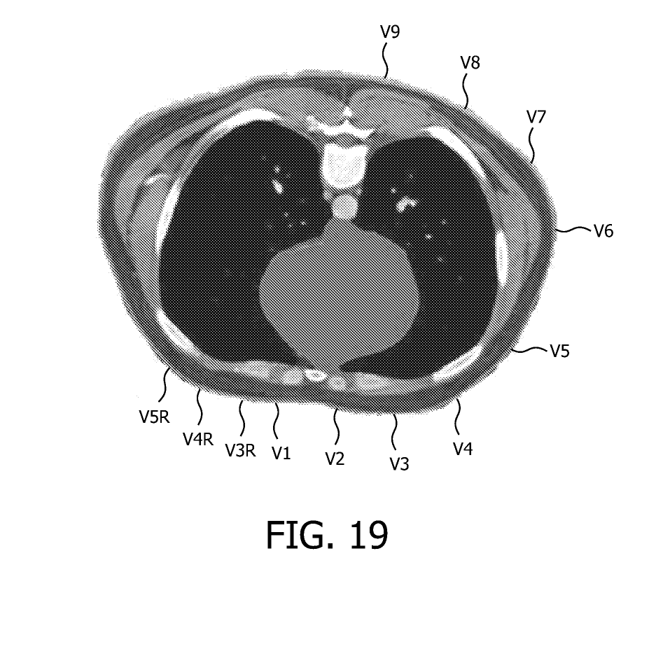

FIG. 19 is a cross-sectional view of a torso showing the relative lead location of an extended set of leads.

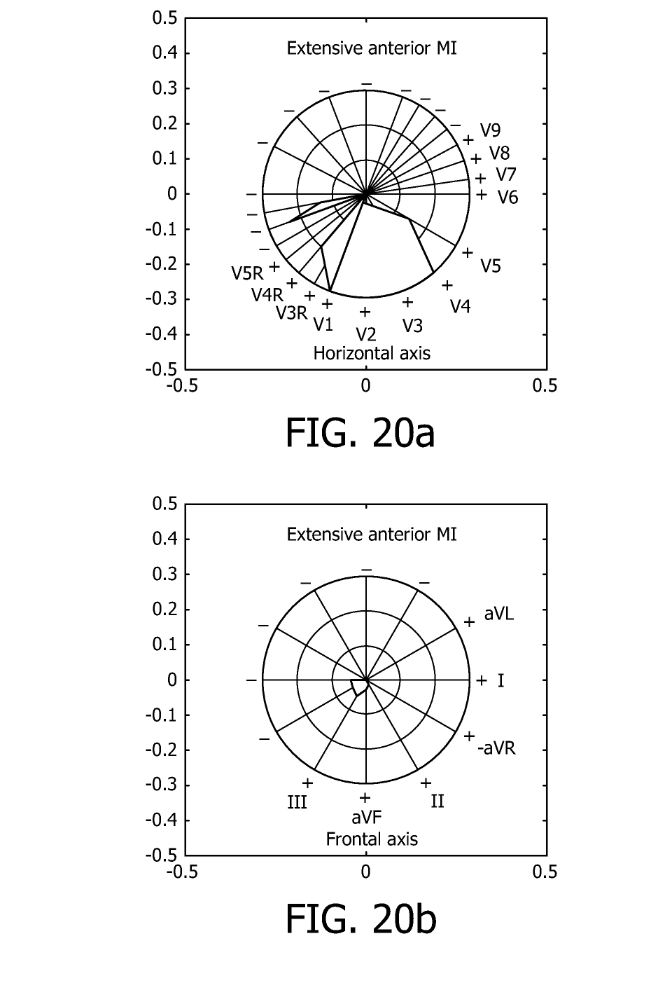

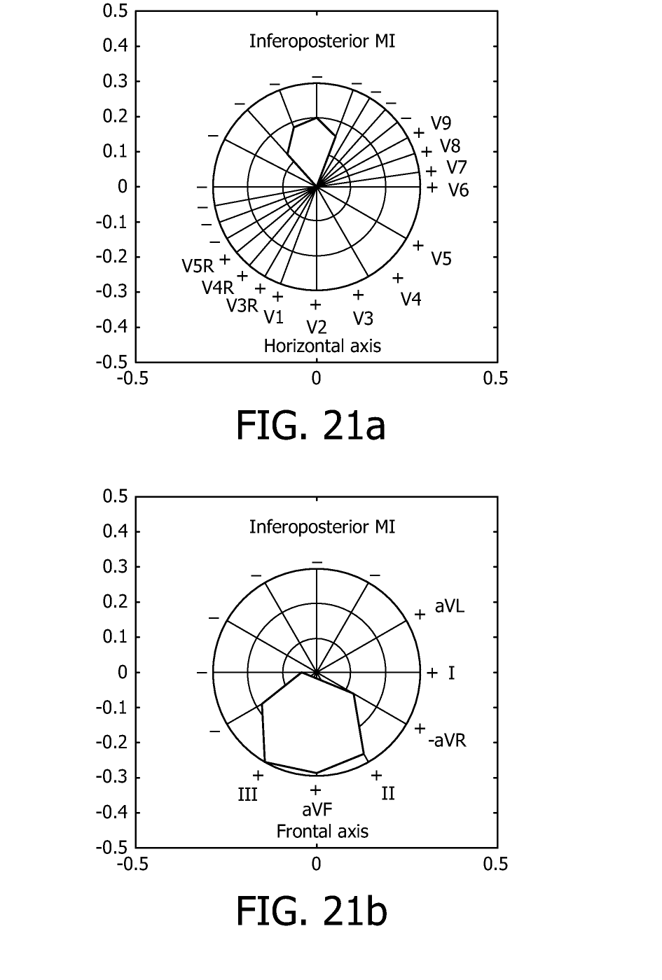

FIGS. 20a and 21a illustrate anatomically-oriented polar displays of the present invention in which the extended leads have been positioned on the polar diagram so as to avoid conflicting with other leads of the diagram.

FIGS. 20b and 21b are frontal axis polar diagrams for the cases of FIGS. 20a and 21a, respectively.

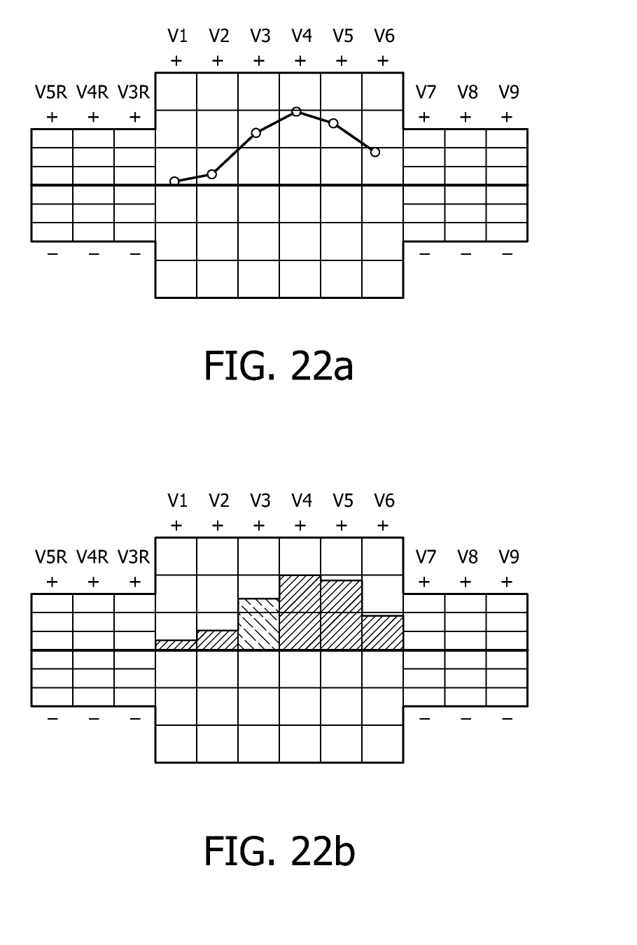

FIG. 22a illustrates a linear graphical display for the horizontal axis of the chest leads.

FIG. 22b illustrates a linear graphical display for the chest leads in the form of a bar chart, with interpolation to fill in missing data.

FIG. 1 is a view of the heart showing the locations of the coronary arteries which, when obstructed, will cause significant damage to the heart. In FIG. 1 the heart 10 is depicted as a translucent orb so that the tortuous paths of the coronary arteries on both the anterior and posterior surfaces of the heart can be readily visualized. The right coronary artery (RCA) is seen descending along the right side of the heart 10 from the aorta. Also descending from the aorta along the left side of the heart is the left main (LM) coronary artery, which quickly branches to form the left anterior descending (LAD) artery on the front (anterior) surface of the heart and the left circumflex (LCx) artery which wraps around the back (posterior) of the heart. All three major vessels are seen to ultimately wrap around the heart 10 in characteristic tortuous paths to provide a constant supply of fresh blood to the myocardium. When a patient is experiencing chest pain due to occlusion of one of the coronary arteries, it is important to quickly identify the arterial branch which is occluded so that intervention can be performed quickly to prevent damage to the heart.

FIG. 2 illustrates the limb leads of a typical ECG system and their relationship to the anatomy of the body. The limb lead signals and the other lead signals of an ECG system are produced by combining the outputs from specific electrodes attached at certain locations on the body. U.S. Pat. No. 6,052,615 (Feild et al.), for instance, shows how the lead signals are developed for a 12-lead ECG system. In the illustration of FIG. 2, the AVR lead relates to the right arm, the AVL lead relates the left arm, and the AVF lead relates to the left leg of the body. When a person is standing as shown in the drawing, these three leads are in approximately a vertical (transverse) plane. For purposes of the present invention the lead signals have a polarity in relation to ST elevation above a nominal baseline as indicated by the “+” symbols in the drawing. At the opposite ends of the axes drawn along the respective limbs the lead signals have a negative connotation for ST elevation. This lead orientation and relationship will be discussed further below in connection with the various illustrations of displays of the present invention.

FIG. 3a shows the placement of six ECG chest electrodes V1-V6, which are located on the torso of the patient. FIG. 3b shows chest electrodes V7-V9 which continue around to the back (posterior) of the patient. As in the case of the limb electrodes, the signal of each chest electrode is used in combination with the signals of one or more other electrodes to detect voltages produced by depolarization and repolarization of individual heart muscle cells. For a 12-lead ECG system the detected voltages are combined and processed to produce twelve sets of time varying voltages. The tracings so produced are described in Feild et al. as follows:

| Lead | Voltage |
|---|---|
| I | VL − VR |
| II | VF − VR |
| III | VF − VL |
| aVR | VR − (VL + VF)/2 |
| aVL | VL − (VR + VF)/2 |
| aVF | VF − (VL + VR)/2 |
| V1 | V1 − (VR + VL + VF)/3 |
| V2 | V2 − (VR + VL + VF)/3 |
| V3 | V3 − (VR + VL + VF)/3 |
| V4 | V4 − (VR + VL + VF)/3 |
| V5 | V5 − (VR + VL + VF)/3 |
| V6 | V6 − (VR + VL + VF)/3 |

The present invention is suitable for use with conventional 12-lead EGG systems as well as with 13-, 14-, 15-, 16-, 17-, or 18-lead or greater systems, including 56- and 128-lead body surface mapping systems. Three-lead (EASI and other), 5-, and 8-lead systems can also be used to derive 12 leads, with reduced accuracy as is known in the art. See, for example, U.S. Pat. No. 5,377,687 (Evans et al.) and U.S. Pat. No. 6,217,525 (Medema et al.) In sum, an implementation of the present invention can employ any number of leads and electrodes.

It can be seen that the chest electrode locations in FIGS. 3a and 3b are in approximately a horizontal plane with respect to a standing individual. As will be discussed below, this anatomical relationship also plays a role in the illustrated embodiments of the present invention.

FIG. 4 illustrates in block diagram form an ECG monitoring system suitable for use with the present invention. A plurality of electrodes 20 are provided for attaching to the skin of a patient. Usually the electrodes are disposable conductors with a conductive adhesive gel surface that sticks to the skin. Each conductor has a snap or clip that snaps or clips onto an electrode wire of the ECG system. The electrodes 20 are coupled to an ECG acquisition module 22 of the monitoring system that preconditions the signals received by the electrodes. The electrode signals are coupled to an ECG processing module 26, generally by means of an electrical isolation arrangement 24 that protects the patient from shock hazards and also protects the ECG system when the patient is undergoing defibrillation, for instance. Optical isolators are generally used for electrical isolation. The processed ECG information is then displayed on an image display or printed in an ECG report by an output device 28.

FIG. 5 shows the acquisition module 22 in greater detail, starting with a signal conditioner 32. The electrode signals, which are usually just a few millivolts in amplitude, are amplified by amplifiers which also usually have high voltage protection from defibrillation pulses. The amplified signals are conditioned as by filtering and then converted to digitally sampled signals by analog to digital converters. The signals are then formatted by differentially combining various electrode signals to derive lead signals in combinations such as those given above for a 12-lead system. The digital lead signals are forwarded for ECG processing under control of CPU 34. Much of the specialized electronics of the acquisition module is often implemented in the form of an application-specific integrated circuit (ASIC).

FIG. 6 is a block diagram of the analysis portion of a typical diagnostic ECG system. A pace pulse detector 42 identifies and sets aside electrical spikes and other electrical abnormalities produced by a pacemaker for patients who are wearing one. A QRS detector 44 detects the dominant pulse of the electrical traces. FIG. 9a illustrates a typical normal ECG trace, where it is seen that the Q-R-S segments delineate the major electrical pulse of the trace, which is the pulse that stimulates a contraction of the left ventricle. Delineation of the QRS complex forms the basis for detecting the lesser perturbations of the trace, which is performed by the waveform segmenter 46. The waveform segmenter delineates the full sequence of trace segments including the P wave and the Q to U segments of the ECG trace including the S-T segment. With each waveform now fully delineated, a beat classifier 48 compares each new beat with previous beats and classifies beats as normal (regular) or abnormal (irregular) for the individual undergoing diagnosis. The classification of the beats enables an average beat analyzer 52 to define the characteristics of a normal heartbeat and the amplitudes and segment durations of an average beat are measured at 54. The beat classifications are used to determine the heart rhythm at 56. FIGS. 7 and 8 are functional illustrations of some of this ECG trace processing.

At the left side of FIG. 7 is a series 60 of ECG traces from leads I, II, V1, V2, V5 and V6. The beat classifier 48 compares the various beat characteristics and has classified some of the beats as normal (N*,0). For example, all of the beats from leads V5 and V6 have been classified as normal. The other four leads contain a beat exhibiting the characteristics of premature ventricular contraction (PVC,1) in this example. At 62 the ECG system aggregates the characteristics of the normal beats, excludes characteristics of the abnormal beats, aligns the beats in time and averages them to produce an average beat. The traces at 64 illustrate the traces of an average beat for the six leads shown in this example. In FIG. 8 the average beat traces 64 of the six leads are measured for various characteristics shown at 66, such as the amplitudes and durations of the Q wave, the R wave, and the T wave and inter-wave intervals such as QRS, ST, and QT. The measurements are illustrated as recorded in a measurement table 68 for the six leads of this example. The ECG waves and their measurements can be sent to an offline workstation with a report generation package for the production of a report on the patient's ECG waveforms. However most diagnostic ECG systems such as the Philips Pagewriter® line of cardiographs and the Philips TraceMaster® ECG management system have onboard ECG reporting packages.

In accordance with a further aspect of the present invention, ECG lead signals are analyzed for particular patterns of elevated and depressed ST segments which relate to stenoses of specific coronary arteries and branches. In the normal ECG trace of FIG. 9a, the signal level of the ST segment 80 is at or very close to the nominal baseline of the ECG trace. When a coronary artery becomes fully occluded, the ST segment 82 for a lead in proximity to the artery will be highly elevated as shown in FIG. 9b, where the dashed line indicates the nominal baseline of the trace. The ST segment can be elevated 100 μvolts or more. ECG leads proximate to the other side of the heart will exhibit a corresponding depression, which can be detected and correlated with the elevated trace for correlating identification of the ST elevation. Furthermore, the amount of ST elevation will vary as a function of the time and degree of stenosis. For example, shortly after the time of the event causing the obstruction, the ST segment of a lead will exhibit a relatively significant elevation 84 as shown in FIG. 9c. With the passage of time the elevation will decrease, and the ST elevation 86 can appear as shown in FIG. 9d. After a substantial period of time, as the heart begins adapting to its new physiological condition, or when an artery is only partially occluded, the ST segment will be only slightly elevated or depressed as shown at 88 in FIG. 9e. ST depression is present when the ST segment is below the nominal baseline of the waveform. Thus, by querying the patient as to the time of onset of the chest pain the time of the event can be noted and the expected degree of elevation assessed. The degree of elevation can also be used to recognize only partially occluded vessels such as those in which an old blood clot has calcified over time. These indications can be used to set aside vessels not needing immediate attention while the interventional procedure is directed to the vessel which has just suffered major obstruction.

In accordance with the principles of the present invention, one of the present inventors has studied the statistical analyses of ECG databases and their relationship to different coronary artery anatomies and has participated in the development of an automated technique to identify the culprit artery of an acute ischemic event as described more fully in the previously referenced Zhou et al. patent application, the contents of which are incorporated herein by reference. This inventive technique can identify one of the two main coronary arteries, the RC and the LM, or one of the two main branches of the LM, the LDA or the LCx, as the culprit artery. The cardiologist is then informed of the identity of the culprit artery as by identifying it in the ECG report, visually on a screen, on a display of ECG traces, audibly, or by other output means. The other inventors have developed an inventive display technique for monitored ECG information as described in international publication number WO 2006/033038 (Costa Ribalta et al.) which is incorporated herein by reference. This display technique presents monitored data in a way that allows rapid detection of data in its spatial situation. Two and three dimensional graphical illustrations are presented in this patent publication. The illustrated graphical displays give information not only about the current values of ST segment data but also about the spatial arrangement of the data. In accordance with the present invention, the present inventors have incorporated aspects of all of these developments to provide an ECG system which presents an anatomically-oriented graphic of ECG data from which a clinician can quickly identify a culprit coronary artery which is occluded and a possible cause of an acute ischemic event. A monitoring system of the present invention can be used with a patient with chest pain who has just arrived at a hospital and needs an initial diagnosis, as well as with patients who have undergone intervention and who are being monitored for further coronary artery occlusions or abnormalities.

Referring now to FIG. 10, a display 100 of the type described in Costa Ribalta et al. is shown. The graphic 102 on the left uses the limb leads which, as previously mentioned are approximately in a vertical plane. In addition to the AVR, AVL and AVF leads shown in FIG. 2, the graphic 102 uses the I, II, and III leads which are also developed from limb electrode signals. The graphic includes axes for the signals which are oriented in relation to the limb positions shown in FIG. 2, with axis for the I lead being the horizontal (0°) axis in the drawing and the II and III lead axes disposed on opposite sides of the vertical (90°) AVF axis. In this example the ends of the axes are scaled to 2 mm of ST elevation, the millimeter notation being familiar to most cardiologists. The translation from the electrical units measured by the ECG system to the millimeter notation is 100 μvolts equals 2 millimeters.

The axes in the graphic 102 are also seen to have + and − polarities. A lead exhibiting an ST elevation will have the data value plotted on the positive side of the axis from the origin, and ST depression measurements are plotted on the remaining negative side of the axis. The graphic 102 is seen to have six ST data values plotted on the axes of the graphic. The value of point 111 on the axis for the II lead, for example, is near the positive end of the axis. This is an ST elevation value approaching 2 mm in the scale of this drawing. The ST elevation value of the AVF lead is also approaching 2 mm as shown by point 113 near the + end of the AVF axis. The point 115 plotted on the AVL axis is seen to be on the negative side of that lead axis. In this example point 115 shows that ST depression of approximately 1 mm is present on the AVL lead.

The points plotted on the lead axes are connected by lines and the area inside the lined shape 112 is colored or shaded as shown in the drawing. Thus, the clinician can see at a glance that the plotted ST values delineate a sizeable shape 112 centered at the bottom of the graphic.

A similar graphic 104 is provided for the chest leads as shown at the right side of the display 100. In this example axes for the chest leads are arrayed from V1 through V6 in the same order as they are physically oriented on the chest. In this example the V1 axis is located at approximately the 112° position of the polar graphic and the other lead axes proceed counter-clockwise from this position. While this example uses only the six leads on the front (anterior) of the chest (FIG. 3a), it will be appreciated that axes for the other chest leads V7-V9 which continue around the torso to the back of the chest as shown in FIG. 3b can also be included to further fill out the array of axes in the graphic 104. This graphic 104 uses + and − polarities for the ST elevation and depression values in the same manner as graphic 102. The ST elevation and depression values are similarly plotted as points on the respective lead axes and the points connected to form a shape 114 in the same manner as graphic 102. Thus it is seen at a glance that the chest lead graphic 104 is presenting a slightly smaller shape 114 which is positioned in the lower right quadrant of the graphic, centered around the V4 lead location.

The display 100 of FIG. 11 is similar to that of FIG. 10 but has been drawn to shown the millimeter values of the ST segment measurements adjacent to the respective lead axes. For instance lead III is exhibiting ST depression of −0.5 mm, which is plotted on the negative side of the III lead axis and defines the greatest extension of the shape 112 from the origin of the polar graphic. The chest lead V4 with a measured ST elevation of 0.6 mm defines the greatest extension of the shape 114 from the origin of the chest lead graphic 104. It is seen that the axes in this example are scaled to a maximum extension of ±1 mm.

In accordance with the principles of the present invention, the locations of the ECG-derived shapes in the anatomically related graphics are used to visually identify suspect culprit coronary arteries. In the limb lead graphic 102 an ECG-derived shape which is located in the region indicated by the circled LAD will generally be symptomatic of obstruction of the left anterior descending (LAD) coronary artery. A shape located around the left center of the graphic is usually indicative of a right coronary artery obstruction as indicated by the circled RCA. Obstruction of the left circumflex coronary artery is signaled by a shape located around the bottom center of the graphic as indicated by the circled LCx. The locations of ECG-derived shapes signaling possible LCx, RCA, and LAD obstruction are similarly shown in the chest lead graphic 104 by the circled letters. The graphic 104 shows an ST segment-delineated shape in the lower right quadrant of the graphic, indicative of obstruction of the left anterior descending coronary artery. It is seen that a clinician can take a quick look at the display 100 and immediately see which coronary artery is the probable cause of an ischemic condition.

The examples below are of anatomically oriented displays indicating obstruction of particular coronary arteries. In FIG. 13 the plotted ST elevation values of the chest leads in the horizontal graphic 104 delineate a sizeable shape 114 in a location of the graphic that is characteristic of LAD occlusion. The limb lead (vertical) graphic 102 shows only a very small shape 112 near the origin of the graphic, showing that virtually no ST elevation or depression has been measured by the limb leads. This display 100 would suggest to a clinician that the LAD is the culprit coronary artery.

FIG. 14 illustrates a display 100 showing both the lead axes and the respective ST elevation or depression measurements plotted on those axes. A sizeable shape 112 is formed in the limb lead graphic 102 by the significant ST elevation values measured for leads II, III, and aVF, and the ST depression values measured for leads I and aVL. Very little ST depression is measured by the chest leads as shown by the small shape 114 in the chest lead graphic 104. As the drawing indicates, the large shape 112 in the lower left quadrant of the limb lead graphic 102 would suggest obstruction of the left circumflex (LCx) coronary artery.

FIG. 15 shows a sizeable shape 112 delineated by ST elevation and depression measurements made at the limb leads and used in the limb lead graphic 102. The location of the shape 112 at the left side of the graphic 102 corresponds to the right side of the patient's anatomy (see FIG. 2). The small shape 114 in the chest lead graphic 104 indicates virtually no ST elevation measured by the chest leads; only slight ST depression. The shapes 112,114 of this display 100 are suggestive of a right coronary artery (RCA) obstruction as indicated by the circled letters over the shape 112.

FIG. 16 is an example of a display 100 which suggests that two coronary arteries are suspect. The shape 112 of the ST elevation data measured by the limb leads in the vertical lead graphic 102 suggests a possible occlusion of the LCx coronary artery. The shape 114 produced by the ST elevation data used in the chest lead graphic 104 is suggestive of a possible occlusion of the LAD coronary artery. This display visually gives the clinician a quick indication that multiple coronary arteries should be examined more closely for possible occlusion.

FIG. 17 is an example of another implementation of the present invention in which the progression of the patient's condition can be monitored. Such an embodiment would be useful, for instance, for a patient upon admission to the hospital with chest pain, when the clinician wants to know if the signs of possible ischemia are increasing. In this display 100 each of the graphics 102,104 shows an outline 122,124 of the shape delineated by ST elevation measurements made at the time that the patient was first connected to the ECG system electrodes. These initial outlines 122,124 may be shown constantly on the display 100 or may be recalled by the clinician. Also shown on the limb lead and chest lead graphics 102,104 are shapes 112,114 delineated by the most current ECG measurements made by the ECG system. By comparing the initial and current shapes 122,124 and 112,114 on the display, the clinician can see at a glance whether the indications of coronary occlusion are increasing, declining, or remaining the same. In this example the shapes 112,114 of the most current measurements are noticeably larger than those of the measurements at the time of admission to the hospital, indicating the possibility of a worsening ischemic condition.

FIG. 18 is another example of an embodiment for monitoring the progress of the patient's condition over time. In this embodiment ST elevation is measured at periodic intervals, in this example, every five minutes. Each time a measurement is made, the outline A, . . . E of the shape delineated by the ST elevation measurements at that time is retained on the display or saved to be called up and displayed as desired. In this example the five successive outlines A, . . . E acquired over time and displayed in the limb lead graphic 102 show a progression indicating an increasingly deteriorating condition of LCx occlusion (see FIG. 12). The five successive outlines A, . . . E displayed in the chest lead graphic 104 indicate a possible progression of LAD coronary artery occlusion. The simultaneous display of the successively produced outlines immediately depict trends of the patient's condition over time. The different outlines may be differently drawn or colored on the display for ease of interpretation.

While the foregoing examples are of displays with two two-dimensional (vertically and horizontally oriented) graphics, it will be appreciated that this information can be combined vectorially into a single graphic display, or in a single three-dimensional display which may be examined and moved or rotated (e.g., dynamic parallax) by the operator to present a three-dimensional impression of coronary artery defects.

In addition to the ST elevation and depression characteristics described above, other ECG measurements such as amplitudes and durations of Q wave, R wave, T wave and interwave intervals such as QRS and QT may also be used as applicable in the identification of the culprit coronary artery. The use of higher order lead sets including 13- to 18-lead ECG systems and 64- and 128-lead ECG body surface maps can provide additional incremental information to enhance the accuracy of culprit coronary artery identification. For systems with fewer than 12 leads, additional lead signals can be derived to implement the technique of the present invention with potentially reduced accuracy. It will also be appreciated that thresholds of ST elevation can be used for different ages, genders, and leads which are determined by appropriate AHA guidelines or other criteria. The graphical display can be highlighted as by coloring or labeling the outlined areas with the identity of the suspected coronary artery when ST elevation measurements exceed the appropriate thresholds for a patient. For instance, an outlined area can be highlighted if a male patient between 30 and 40 years of age presents ST elevation in leads V2 and V3 of greater than 2.5 mm (250 μvolts) and ST elevation in excess of 1 mm (100 μvolts) for all other leads. For a female, the area would be highlighted if ST elevation in the critical leads exceeds 1.5 mm (150 μvolts). Other threshold criteria may be used as appropriate standards are developed.

The foregoing graphic displays of FIGS. 10-18 plot lead values for a standard ECG lead set, where ten electrodes are used to acquire and calculate the twelve standard lead signals. However extended lead sets are sometimes used for a patient which acquire and produce a greater number of lead signals. This is illustrated by FIG. 19, which is a cross-sectional view through the chest at the heart level and shows the use of additional posterior leads V7, V8, and V9 and additional frontal leads V3R, V4R, and V5R from additional chest electrodes. Ambiguities can arise in the graphic displays when these extended lead values are plotted together with the standard lead values. These ambiguities result from the electrical forces produced by the heart (the light shaded region at the bottom center of the cross-section) which are subject to a phenomenon known as reciprocal changes. As the locations of the leads indicate, several pairs of leads oppose each other on opposite sides of the body. For example, the V9 lead is opposite the location of the V2 lead. By virtue of this orientation, the voltages of the opposing electrodes will be polar opposites of each other. When the voltage of V9 is positive, the voltage of V2 will be negative. As previously mentioned, when the V2 lead is experiencing ST elevation, the V2 voltage will be plotted as a positive signal on the V2-symbol side of the V2 axis of the polar diagram, and when the V2 lead is experiencing ST depression, a negative voltage results which is plotted on the opposite side of the origin on the V2 axis of the diagram. But when V9 voltages are plotted on the same axis, with +V9 vectorially located on the opposite (negative) side of the same axis, the reciprocal changes of the plotted voltages will amplify or possibly cancel each other. This is a problem which is particular to the extended precordial leads which are arrayed in substantially the same horizontal plane around the chest of the patient. It is desirable to prevent this ambiguity from arising while continuing to preserve the true anatomical relationship of the vectorial axes of the polar diagram.

A solution to this problem is shown by the polar diagram of the horizontal axis of FIG. 20a. In this example the extended leads are plotted on vectors which do not directly oppose the vectors of the other (standard) leads. Effectively, the extended leads V7, V8, and V9 have been rotated clockwise to about the 60°, 70°, and 80° positions on the polar diagram. When so located, the extended lead vectors no longer are opposite the vectors of other leads. In particular, it is seen that the V9 vector no longer opposes the V2 vector. Similarly, the extended leads V3R, V4R and V5r are positioned at approximately the 210°, 220°, and 230° axes of the polar diagram. Thus, each lead vector is on its own axis and it is clear what value was recorded from each electrode. This may be due to reciprocal changes but what is displayed is what was recorded.

Furthermore it is seen that all of the positive lead orientations are in one-half of the polar circle, and the negative halves of their axes are all positioned in the other half of the circle. In this example the positive ends of the axes are all in approximately the 55°-235° half of the polar diagram and the negative ends are all in the 235°-55° half of the diagram. Thus the differentiation of plotted values for ST elevation and ST depression can be immediately perceived by a viewer.

In the exemplary polar diagram of FIG. 20a, it is seen that positive elevated values are plotted on the diagram for the V3R and V4R leads, extending the graphical area defined by values for leads V1-V5. Negative values for leads V6, V7, and V8 create a second smaller outlined area on the diagram. As is stated on the polar display, these graphics and their elevated ST values are symptomatic of extensive anterior myocardial infarction. The corresponding frontal axis diagram for the same patient is illustrated in FIG. 20b.

FIGS. 21a and 21b are exemplary polar diagrams for a patient with inferoposterior myocardial infarction. The horizontal axis polar diagram of FIG. 21a shows a negative value for extended lead V3R, extending the graphical area defined by negative V1-V5 lead values. A second, smaller area is defined by elevated values of the V6-V8 leads. The frontal axis graphic is shown in the polar diagram of FIG. 21b.

FIGS. 22a and 22b show linear or rectilinear, rather than polar, presentations of the lead values. Both of these diagrams are shown for the precordial (chest) lead of the horizontal plane, although similar linear diagrams can also be used for the vertical (limb lead) plane. In these two drawings leads V1-V6 are calibrated in large boxes around a center (zero) line, with each box corresponding to 100 μvolts in the vertical direction. The extended leads V5R, V4R and V3R, and V7-V9 are calibrated in small boxes, each corresponding to 50 μvolts in the vertical direction in keeping with the relative magnitudes of these lead signals. In FIG. 22a the lead values of FIG. 10 have been plotted in each lead column and connected by a line, with the area under the line indicating the culprit artery. In other embodiments the individual lead signals can be plotted in rows rather than columns.

FIG. 22b is similar to FIG. 22a, except that instead of plotting the lead values as points or circles, the lead columns of the display are filled to the level of the lead signal values in the manner of a bar chart. In this example it is further assumed that the values of lead V3 is missing from the lead data. The cross-hatching of the other lead columns with solid lines representing colors or shading that indicates actually received and computed lead signals. The dashed cross-hatching of the V3 lead column represents a different color or shading that indicates to the viewer that this lead value is missing from the data set but has been estimated based on other lead data. In this example the level of the shading of the V3 column is an average or interpolation of the adjacent (V2 and V4) lead values. The build-up of the shaded columns again points to the culprit artery, and with a display that shows the viewer that the V3 lead data was missing and has been estimated.

Other graphical display formats for plotting the lead data will readily occur to those skilled in the art. For example, the polar display may use shading or colors to indicate missing leads in like manner. Another variation is to color outlined areas of the polar display within the polar range of extended leads with different colors or shading than areas defined by the standard lead values.

What is claimed is:

1. An ECG monitoring system which identifies a culprit coronary artery associated with an acute myocardial infarction comprising:
a set of electrodes adapted for acquisition of electrical activity of the heart from different vantage points in relation to the heart, the set of electrodes including extended leads in addition to standard leads;
an ECG acquisition module coupled to the electrodes which act to produce enhanced electrode signals;
an ECG processor responsive to the electrode signals and configured to combine electrode signals for the production of a plurality of lead signals measuring electrical activity of the heart from different vantage points, wherein the ECG processor is further configured to detect ST elevation in lead signals; and
a graphic display responsive to detected ST elevation which displays ST elevation data graphically in relation to anatomical lead locations, with elevation data from different leads located in locations which do not interfere with each other,
wherein the graphical display indicates the identity of a suspect culprit coronary artery or branch associated with an acute ischemic event.

2. The ECG monitoring system of claim 1, wherein the ECG processor is responsive to a plurality of chest electrode signals for the production of ST elevation data and use of the data for the production of a chest graphic oriented horizontally with respect to the anatomy of a subject,
wherein the chest graphic is indicative of one or more of LCx, RCA, and LAD coronary artery occlusion.

3. The ECG monitoring system of claim 2, wherein the chest graphic further comprises a shape delineated by ST elevation data values.

4. The ECG monitoring system of claim 3, wherein the shape is formed by connecting ST elevation data values in the chest graphic.

5. The ECG monitoring system of claim 3, wherein ST elevation data values are located with one polarity in the chest graphic, and ST depression data values are located with an opposite polarity in the chest graphic.

6. The ECG monitoring system of claim 1, wherein the graphic display further comprises a polar diagram with different lead signals plotted on different polar vectors.

7. The ECG monitoring system of claim 6, wherein positive lead signal values are plotted in one-half of the polar display and negative lead signal values are plotted in the other half of the polar display.

8. The ECG monitoring system of claim 7, wherein the positive lead signal values comprise ST elevation values and negative lead signal values comprise ST depression values.

9. The ECG monitoring system of claim 6, wherein an estimated value is plotted for a missing lead signal.

10. The ECG monitoring system of claim 1, wherein the graphic display further comprises a linear or rectilinear diagram with different lead signals plotted on different rows or columns.

11. The ECG monitoring system of claim 10, wherein each row or column with a lead signal value indicates a lead signal magnitude and is connected to the lead signal magnitude of one or more adjacent rows or columns.

12. The ECG monitoring system of claim 10, wherein each row or column with a lead signal value is filled to a lead signal magnitude level with a color or shading.

13. The ECG monitoring system of claim 10, wherein an estimated value is plotted for a missing lead signal.

14. The ECG monitoring system of claim 13, wherein the estimated value is an interpolation or average of other received lead signal values.
